(19) United States
(12) Patent Application Publication
(10) Pub. No.: US 2008/0085839 A1
(43) Pub. Date: Apr. 10, 2008

(54) Method For The Analysis Of Point Mutations

(76) Inventor: Holger Klapproth, Freiburg (DE)

Correspondence Address: THE WEBB LAW FIRM, P.C., 700 KOPPERS BUILDING, 436 SEVENTH AVENUE, PITTSBURGH, PA, 15219, US

(21) Appl. No.: 11/661,563

(22) PCT Filed: Sep. 1, 2004
(86) PCT No.: PCT/EP04/09730

§ 371 Date: Feb. 28, 2007

(51) Int. Cl. C12Q 1/68; C40B 30/10; C40B 50/04

(52) U.S. Cl. 506/12; 435/6; 506/25

(57) ABSTRACT

The invention relates to a method for determining point mutations by means of DNA microarrays and TIRF excitation. According to said method, bonding of nucleic acids to short DNA probes on a microarray is measured at different temperatures. Melting point curves are generated from the measured values and the difference in the melting point curves between the probe for the wild-type DNA and the probe for the corresponding mutated DNA is generated. The position of said curves makes it possible to unambiguously decide whether the point mutation is a homozygous DNA or a heterozygous DNA and what type of homozygosity it is.

### THE PRIOR ART

In many analyses of genomes, for example, of the human genome, so-called single nucleotide polymorphisms (SNPs) play a key role. Many genetic diseases, for example, hereditary hemochromatosis, are linked to such SNPs. In the simplest case, such SNPs can be detected by PCR or by the sequencing of the respective gene. If the zones of interest, however, are separated from each other by more than a few hundred base pairs, then sequencing is not so easy or at least becomes considerably more expensive. Furthermore, with more complex mutations, it is possible that zones of the gene are no longer capable of being analyzed in the DNA sequencer. If there are several possible SNPs on a gene, the PCR analysis becomes very difficult and once again very expensive.

There have been efforts to parallelize the analysis of SNPs for some time. An example of such a method is represented by the oligonucleotide microarrays. Diverse DNA fragments (oligonucleotides) are bonded to said microarrays, wherein the base that is complementary to the target SNP is located in the middle of the strand in said fragments. Based on the resulting differences in the melting points from the analyte DNA, it is now possible to confirm whether 100% complementary DNA has bonded or not. The usual indication lies in the comparison of the signal intensities between the perfect matches (100% complementary) and the SNP mismatches (ca. 5% misbonding). These analyses were usually performed by the hybridization of the DNA on the chips at a fixed temperature, and then the bonding intensity was measured and compared. If a plurality of SNPs is analyzed simultaneously on one chip, it is nearly impossible to adapt all of the probes to the same analysis temperature, especially because the methods for calculating the melting point for DNA hybrids do not correspond to the measured values.

For this reason, better methods for accurately measuring large quantities of SNPs are needed. Such methods are based mainly on the definition of the melting point of the hybrids on the chip. Devices with which said analyses can be performed in real-time are ideally suited for this purpose; the ATR reader developed by the Frauenhofer Gesellschaft (FGH-IPM) is an example of such a device (see FIG. 1). The ATR reader analysis method, however, is relatively complex in that the melting curves of the nucleic acids are analyzed herein in order to determine the so-called melting points (Tm) of the nucleic acid hybrids. To this end, the melting points of DNA probes must be calculated and then experimentally confirmed. It is this technique in particular that makes the design of chips with a plurality of probes per chip relatively difficult.

### DESCRIPTION OF THE INVENTION

The invention describes the surprising finding that no probe design in the true sense is necessary for the analysis of SNPs on ATR chips; instead it is sufficient to incorporate said probes in a prespecified length, which preferably does not differ too much from probe to probe, and then record the melting point curve in each case. The curves of the wild-type probes, henceforth also designated as wt probes, are then correlated with the probes of any mutants. This correlation is achieved by the subtraction of the melting curve of the wild-type probe from the melting curve of one or a plurality of mutations. The result is presented as a graph (see FIG. 2). The position of this curve indicates which genotype is present. An accurate classification, however, is achieved by correlation with reference data. This leads to decisive advantages in the analysis, especially when sequences are used that are not suitable for analysis on a normal DNA microarray. The detection accuracy for point mutations is significantly increased and the analysis is very easy to automate. Furthermore, it is possible to develop chips much more rapidly, as no adaptation to parallel analyses, which function at a fixed hybridization temperature, takes place. It is thus sufficient to calculate the probes with a fixed length and immobilize (or synthesize) them on the ATR chip.

The base (mutation) to be analyzed is preferably located in the middle of the DNA probe sequence. When a chip produced to these specifications is used, all that is then necessary is to hybridize the sample DNA, which preferably must be labeled with a fluorophore, on said chip. Said chip is then heated in 1° C. increments from, say, 30° C. to 80° C. After each temperature increment, the signal on each probe is recorded and stored. After the attainment of the upper selected temperature, a complete melting curve is consequently recorded.

The data for the variations (mutations) to be examined are then correlated with the data of the so-called wild-type sequence. For this correlation, the data of the variations are subtracted from the data of the wild-type sequence. If the analyzed DNA is homozygous for the wild type, the resulting curve will have positive values, at least significantly more positive values than for a heterozygous sample, in the zone of the melting point of the DNA. If the sample is heterozygous, the resulting curve will then ideally approach a straight line along the zero point. If the sample is homozygous for the mutated DNA, then the melting point on the probe of the mutated DNA (there is 100% bonding) is higher than the melting point on the probe for the wild type (there is no 100% bonding). Consequently, the curve, which is calculated by subtracting the melting curves, is characterized by a deflection toward the negative y-axis. The position of these curves explicitly describes the composition of the sample DNA relative to the mutation to be examined.

An examination of this nature preferably takes place in an apparatus such as that described, for example, in EP000124898. Preference is given to the sensors being waveguide chips, which can be slide-shaped chips in which the excitation light is injected across an angle for a TIRF excitation. Chips configured as thin film waveguides are also an option. Such thin film waveguide chips are, say, tantalum pentoxide-coated glass slides in which the light is injected via optic grids. DNA probe molecules are preferably bonded to such chips by chemical adhesive agents such as silanes or polymer coatings. Examples of such compounds are described in EP 1 132 739 B1. Examples of methods for coating carriers with polymer layers are described in EP 1 176 422 A1. Suitable DNA probes for bonding to such surfaces are preferably equipped with a polythymidine spacer comprising at least 10 thymidine molecules. The probe molecules are preferably covalently bonded. To this end, suitable methods are described in WO 00/43539 A2. Examples of the manufacture of suitable samples and labeling are described in WO 01/53822 and in Lehr (Lehr 2002).

In another embodiment of the invention, the surface of the carrier is coated with a UV reactive polymer. Said polymer is then covalently bonded to the surface by irradiation of said coated carrier with UV light. Such thin polymer layers can be applied to polymer carriers by dip coating in a dilute solution (along the lines of EP 1 176 422 A1). DNA molecules can be applied to said surfaces by means of polythymidine spacers and UV crosslinks. Special preference herein is given to copolymers with a limited hydrophily, negatively charged groups, and benzophenone groups for the bonding of the polymer layer to the surface. A polymer composed of metacrylic acid, styrene, and benzophenone methacrylate, for example, is equipped with these properties. Other copolymers and especially block copolymers are obviously also suitable. If said polymer layers are to be applied to glass or other inorganic substrates, it is practical to silanize said substrate beforehand in order to improve the adhesion of said polymer layers. An example of such a silanization is described in EP 1 132 739 B1 and EP 1 176 422 A1.

Suitable carriers for analysis can also be integrated systems (DE 10 245 435 A2, for example, CMOS chips with accessory waveguide structures attached). Said waveguides are preferably located above the optosensors and bonded to the optochip via a thin layer comprising a substrate that has an optic density lower than that of said waveguide.

In another preferred embodiment, the biomolecules are attached to polymer-polymer thin film waveguides. Such waveguides consist of a carrier polymer with a low optic density and a waveguide polymer with a high optic density, such as, for example, a polymer used for the manufacture of plastic lenses for eyeglasses. Suitable polymers of correspondingly low optical density are sufficiently known to the professional.

In another preferred embodiment, the chip is rinsed with a buffer after a measurement and heated so that the bonded nucleic acids are largely removed. Afterwards, the chip can be reused. In addition to heating, the chip can also be regenerated with denaturing agents, such as 0.1% NaOH. This method also makes it possible to hybridize the chip with reference nucleic acids in order to calibrate each chip before, during, or after the measurement. In this way it is possible to correlate the measurements from different experiments with each other and cancel out individual differences, such as those that may arise during the manufacture of the chip. Furthermore, this method enables quality control of the chip during use and makes it possible to determine whether a chip is still suitable for other experiments.

If chips in which the initial hybridization cannot be completely dissolved are used in such experiments, such chips may become saturated by blocking with an unlabeled sample that bonds to all of the nucleic acid probes, so that no residual fluorescence signal remains after the actual experiment and the dissolution of the hybridized samples.

### EXAMPLES

Detection of the H63D mutation of the hemochromatosis gene, Mutation H63D: The base exchange of a cytosine with a guanine (C→G; transversion) on position 187 in exon 2 of the HFE gene leads to an amino acid exchange of a histidine with aspartic acid on position 63 of the HFE protein. This mutation is known as H63D in the literature (Feder et al., 1996).

The H63D mutation involves a transversion, with the exchange of a cytosine with a guanine. A pyrimidine is exchanged with a purine base group in this transversion, and this leads to a steric inhibition on the site of the base mispairing. Because no bond can form in the base mispairings that occur with transversions, the mispairings are more readily detectable.

The probes for detecting the H63D mutation bear a C (wt probe) or a G (mut probe) on the central site of the specific sequence. The sample DNA bears a G (wt sample; H63D (+/+)), a C (mut sample; H63D (−/−)) or 50% G and 50% C (het sample; H63D (+/−)) on the corresponding site.

In the detection, the following base pairings or mispairings occur on the central site of the probe pairs:

In hybridization with a wt sample:

| | |
|---|---|
| wt probe-wt sample: | C≡G |
| mut probe-wt sample: | G G |

In hybridization with a mut sample:

| | |
|---|---|
| wt probe-mut sample: | C C |
| mut probe-mut sample: | G≡C |

In hybridization with a het sample: (about the same signal intensity on wt and mut probes)

| | |
|---|---|
| wt probe-wt sample (and wt probe-mut sample): | C≡G (and C C) |
| mut probe-mut sample (and mut probe-wt sample): | G≡C (and G G) |

1) Manufacture of the Sensor Chips

Oligonucleotides are imprinted on PMMA carriers with dimensions of 76×25×1 mm and a lateral angle of 70° for the injection of the excitation light by means of a Top Spot printer or other suitable device (Lehr 2002). A 5% DMSO aqueous solution is used as a printing buffer. The oligonucleotides are present therein in a concentration of 10 μM. Said oligonucleotides comprise a detection sequence of the target DNA with a length of 17 nucleotides and with the target mutation in the center of the sequence. A polythymidine strand of at least 10 nucleotides in length is located on the 5′ end of said oligonucleotide. In order to immobilize said oligonucleotides, the freshly printed chips are irradiated for 10 minutes in a UV crosslinking oven (Stratalinker, Stratagene) with UV light having a wavelength of 260 mm, then thoroughly rinsed with water to which a detergent was added (0.1% SDS), and finally rinsed with distilled water and blown dry in a stream of nitrogen.

2) Melting Curves for the H63D Mutation

Chips with a 17mer probe pair (63_{—}17 wt and 63_{—}17mut) were manufactured for measuring the melting curves to detect the H63D mutation. Said chips were hybridized and dyed in the TIRF measuring apparatus with DNA samples having the various characteristics (a) wt sample, b) mut sample, c) het sample), and the dissociation behavior of the probe pair was measured. The fluorescence intensities on the wt- and mut probes were observed over a temperature spectrum ranging from 20° C.-80° C. in 2° C. increments and illustrated in the following diagrams.

3) DNA Purification from Blood

The DNA was purified from 10 ml human blood with the “nucleon extraction and purification kit” from Amersham Biotech.

4) Determination of the Nucleic Acid Concentration

For the precise determination of nucleic acid concentrations, the absorption of UV radiation with wavelengths of 260 and 280 nm by a nucleic acid solution was measured in a spectrophotometer as a function of the respective solvent. According to this determination, an A260 unit corresponds to a double strand DNA concentration of 50 μg/ml and a single strand DNA concentration of 33 μg/ml. The ratio of the absorptions at 260 and 280 nm is an indicator of the purity of the DNA. With optimum purity, it assumes a value of 1.8.

5) Amplification and Labeling of the DNA

Polymerase Chain Reaction (PCR) (Mullis et al., 1986). With the aid of PCR, it is possible to replicate a specific section between two known sequences from a small quantity of DNA. To this end, two oligonucleotide primers that bind diametrically opposed on the complementary strands of a known DNA sequence are required. Furthermore, in addition to the sequence to be amplified, the nucleotides dATP, dCTP, dGTP and dTTP as well as the Taq polymerase that is capable of extending the DNA strand outward from the primers must be added.

The fundamental steps of PCR are:

Denaturization phase: The two stranded DNA is uncoupled to single strands at temperatures of 94-95° C.

Annealing phase: The primers attach to the single stranded DNA at a temperature that approximates the melting temperature Tm of said primers.

Extension phase: At 72° C., the extension of the primers takes place, which results in the synthesis of a double strand of DNA with the help of the Taq polymerase and the added nucleotides. It is possible to produce a very large number of copies of the desired sequence section by repeating these 3 steps up to 50 times.

6) Biotin Labeling of the PCR

Modified nucleotides in addition to the naturally-occurring nucleotides can also be incorporated in the PCR. Fluorescent dyes or biotin, for example, can be attached to said nucleotides, wherein biotinylated nucleotides are capable of being incorporated much more easily and efficiently than, say, Cy5 labeled nucleotides. Biotin-16-dUTP was incorporated instead of dTTP in the DNA in order to be able to detect the PCR product on the chip with streptavidin Cy5. Cy-5 dUTP may also be directly incorporated, as desired.

7) PCR preparation for the biotinylated multiplex PCR to detect hemochromatosis preparations in a volume of 25 ul. A standard program for short DNA amplicons was used for the amplification:

| Template DNA | 100 | nM |
|---|---|---|
| Primer1 Fw | 0.5 | μM |
| Primer1 Rev | 0.5 | μM |
| Primer2Fw | 0.5 | μM |
| Primer2Rev | 0.5 | μM |
| dNTP's (without dTTP) | 0.1 | mM |
| dTTP | 0.08 | mM |
| Biotin dUTP | 0.06 | mM |
| MgCI2 | 2.5 | mM |
| 10× buffer | 1:10 dilution | |
| HotStarTaq | 0.25 | U |

7) Production of Single DNA Strands [sic (Numbering)]

PCR products manufactured with a thioate-modified primer and an unmodified primer were used for the production of single DNA strands. Said PCR products were transferred to a volume of 25 μI with 1 μI T7 gene 6 exonuclease from the Amersham company and incubated at 37° C. for 30 min. The preparation was heated at 85° C. for 10 min in order to stop the reaction.

8) Hybridization

Hybridization was achieved by the addition of the amplified sample in hybridization buffers onto the measuring field of the sensor chip. The hybridization solution was placed directly in the TIRF measuring apparatus in a hybridization cuvette. The carriers were then rinsed with commercially available wash buffers in the hybridization cuvette of the TIRF apparatus for a few seconds.

9) Dying

The dying of the bonded biotinylated PCR fragments was achieved by incubation with a streptavidin-Cy5 conjugate. The stock solution was assimilated in buffer dyes, according to the manufacturer's instructions. This solution was placed directly in the hybridization cuvette of the TIRF measuring apparatus.

10) Measurement of Fluorescence

The carrier was irradiated with laser light in the analyzer. According to the manufacturer's instructions (Amersham Biotech), the Cy5 fluorescent dye used (see FIG. 1) absorbs at a λmax=649 nm and emits at a λmax=670 nm. The emitted light was captured with a CCD camera and the resulting image with the individual fluorescence points was analyzed. The graphic data of the fluorescence intensities were converted to numerical data with a commercially available computer program.

11) Analysis of Melting Curves

All of the steps for the analysis of the melting curves were carried out in the hybridization cuvette in the analytical apparatus. The hybridization of the amplified, biotinylated, single strand DNA sample took place at 20° C. for 15 min to 2 hr on the sensor chip probes. The sample with higher hybridization efficiency was bonded to the chip under these less stringent conditions. In the subsequent dying reaction, the biotinylated sample DNA fragments bonded on the probes were dyed for 5 min with a streptavidin-Cy5 solution. A plurality of individual images was then scanned as the temperature was gradually raised.

### DESCRIPTION OF THE DRAWINGS

FIG. 1 describes the TIRF measurement optics. Laser light from a semiconductor laser diode (1) is focused (3) through a lens (2) and beamed on the injection angle of a TIRF measurement chip (4). Biomolecules (5, 6) labeled with a luminescent dye are located on the surface of said chip. In the evanescent field, said luminescent dyes are excited and emit luminescence light (5, 7), which is capable of being captured by suitable detectors. Luminescent dyes [luminescent dyes was repeated unnecessarily in the source text] located outside the evanescent field are not excited (6) and consequently do not emit light. The fluorescent light (7) can be recorded with commercially available CCD or CMOS cameras.

FIG. 2 shows the analysis according to the invention of three experiments in comparison with each other. According to said analysis, the temperature in ° C. is plotted on the abscissa and the signal difference on a 17mer probe pair (H63D mutation hybridized with various genotypes) is plotted on the ordinate. For the upper curve, a DNA containing only wild type alleles was used as the analyte. A difference curve was obtained by correlating the melting curves that were recorded on the probes for the wild type DNA and the mutated DNA to be examined. To obtain the aforementioned, the hybridized chip is heated from 20° C. to 80° C. in 2° C. increments. Upon attainment of the temperature, the chip is scanned with the camera and the image of the fluorescence is saved. The signal of the fluorescence images of various temperature steps is then analyzed. To this end, the signal intensity on the points on which the probes are immobilized is quantified. Reference points on the chip, on which points the fluorescent-labeled DNA is immobilized, are simultaneously analyzed. This signal diminishes as the chip is heated (because the fluorescence signal is reduced by heat). This decrease is defined as a percent and the recorded signal curves are corrected for this decrease. After the correction has been made, the signal of the probe of the target mutation is subtracted from the probe of the wild type DNA. The result is plotted as a graph. The middle curve represents the case when wild type DNA as well as mutated DNA are present in the analyte. In the ideal case, the curve forms a straight line along the zero point. The bottom curve represents the case in which only mutated DNA is present. The difference of the bonding curve of wild type DNA relative to the bonding curve of mutated DNA is negative, because the mutated DNA bonds to the wild type probe with substantially less bonding energy than it does to the probe of the mutated DNA sequence.

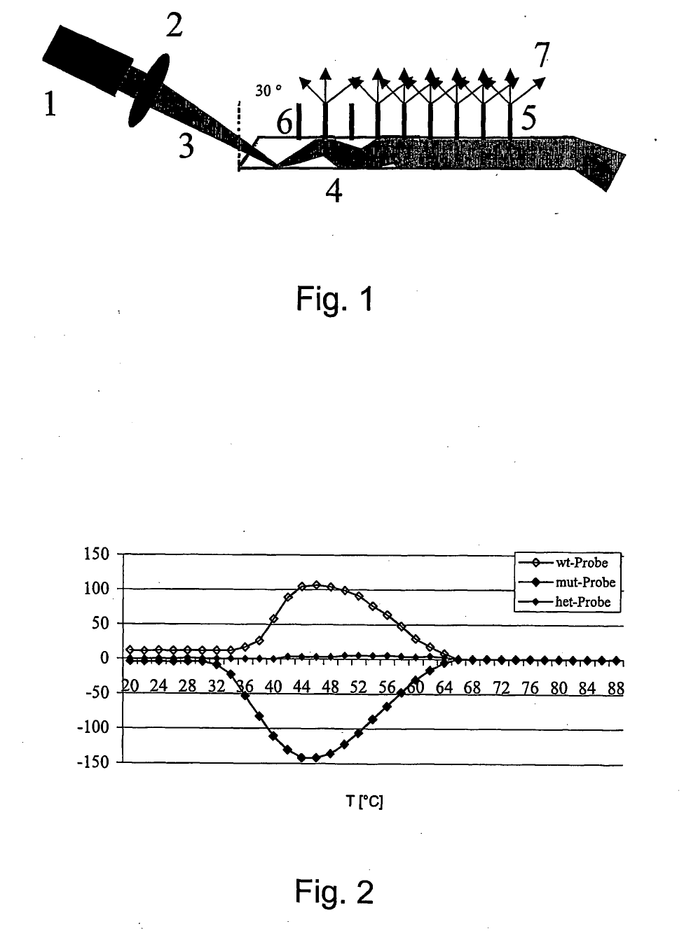

1. A method for the analysis of nucleic acids, comprising:
(a) measurement of the melting curve of a nucleic acid hybrid of a sample nucleic acid with a first nucleic acid probe,
(b) measurement of the melting curve of a nucleic acid hybrid of a sample nucleic acid with a second nucleic acid probe, which differs from said first nucleic acid probe by at least one nucleotide, but which is at least 80% homologous with said first nucleic acid probe,
(c) mathematical correlation of the melting curve from (b) with the melting curve from (a), and
(d) correlation of the result from (c) with reference values for the possible compositions of the nucleic acid samples.

2. The method of claim 1 comprising the further step of
(e) confirmation of a finding on the basis of the collected data.

3. A method for the determination of the identity of a nucleic acid, wherein:
(a) the melting point curve of the bond of a reference nucleic acid of an identical sequence is recorded on a nucleic acid probe,
(b) the melting point curve of the nucleic acid to be examined is recorded on a nucleic acid probe of an identical sequence, and
(c) both of the melting point curves are mathematically correlated.

4. The method according to claim 1, wherein the data are acquired by means of a heatable TIRF apparatus.

5. The method according to claim 1, wherein said nucleic acid probes are covalently bonded to the surface of a waveguide.

6. The method according to claim 1, wherein said nucleic acid probes are covalently bonded to a polymer or a polymer layer.

7. The method according to claim 5, wherein a plurality of nucleic acid probes is attached to the waveguide chip.

8. The method according to claim 5, wherein said waveguide chip is freed from the bonded nucleic acids after the performance of an experiment and used for at least one other experiment.

9. The method according to claim 5, wherein said waveguide chip is hybridized with a reference nucleic acid, in order to obtain correlation values for the subsequent measurements.

10. A method for the reduction of unspecific signals on DNA chips, wherein,
(a) a DNA chip with an unlabeled sample, which bonds on said nucleic acid probes on said chip, is hybridized,
(b) the hybridization is then dissolved, and
(c) said DNA chip is then used in at least one other hybridization experiment with labeled DNA.

11. The method of claim 3, wherein the data are acquired by means of a heatable TIRF apparatus.

12. The method of claim 3, wherein said nucleic acid probes are covalently bonded to a surface of a waveguide.

13. The method of claim 3, wherein said nucleic acid probes are covalently bonded to a polymer or a polymer layer.

14. The method of claim 12, wherein a plurality of nucleic acid probes is attached to the waveguide chip.

15. The method of claim 12, wherein said waveguide chip is freed from the bonded nucleic acids after the performance of an experiment and used for at least one other experiment.

16. The method of claim 12, wherein said waveguide chip is hybridized with a reference nucleic acid, in order to obtain correlation values for the subsequent measurements.
